(19) United States
(12) Patent Application Publication
(10) Pub. No.: US 2006/0167313 A1
(43) Pub. Date: Jul. 27, 2006

(54) Process for preparing encapsulated metalloporphyrin catalyst and process for oxidation of alcohols

(75) Inventors: Rajan H. Naik, Maharashtra (IN); Padmakar L. Joshi, Maharashtra (IN)

Correspondence Address: NIXON & VANDERHYE, PC, 901 NORTH GLEBE ROAD, 11TH FLOOR, ARLINGTON, VA, 22203, US

(73) Assignee: COUNCIL OF SCIENTIFIC AND INDUSTRIAL RESEARCH, New Delhi (IN)

(21) Appl. No.: 11/331,358

(22) Filed: Jan. 13, 2006

Related U.S. Application Data
(63) Continuation-in-part of application No. 11/022,920, filed on Dec. 28, 2004, now abandoned.

(51) Int. Cl. C07C 51/27; C07D 487/22; B01J 31/02

(52) U.S. Cl. 562/540; 540/145; 502/162

(57) ABSTRACT

The present invention relates to a polystyrene encapsulated catalyst containing metalloporphyrin of formula I: 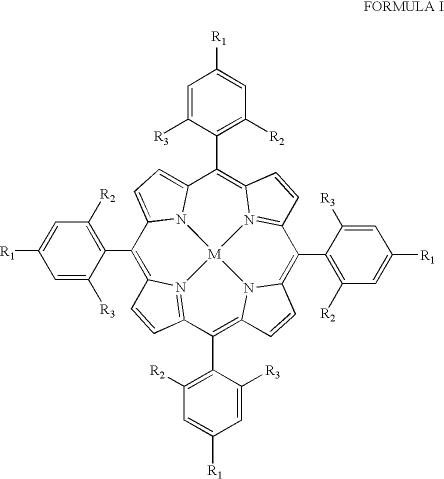
wherein R₁, R₂, and R₃ are individually selected from the group consisting of alkyl, halo, nitro and amino groups and M is a metal atom selected from the group consisting of Fe, Co, Ni, Mn, Cu, Ru, Pt and Pd and to its use in oxidation of alcohols.

This application is a continuation in-part of application Ser. No. 11/022,920, filed Dec. 28, 2004, pending; the entire content of which is hereby incorporated by reference in this application.

### FIELD OF INVENTION

This invention relates to a process for the preparation of encapsulated metalloporophyrin catalyst. More particularly it relates to the process for preparation of the said catalyst containing metalloporphyrins of formula (I) herein below: 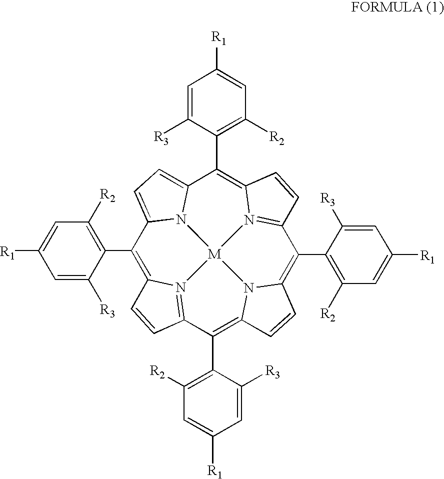
wherein

R₁═H, Cl, Br, CH₃, OCH₃, OH, NO₂, NH₂

R₂═H, Cl

R₃═H, Cl

M=Fe, Co, Ni, Mn, Cu, Ru Pt, Pd etc.

The invention particularly relates to a process for encapsulation of metalloporphyrins of formula (I) in polystyrene matrix. The various porphyrins were prepared as per the reported procedure (Rajan Naik et al, Tetrahedron, 2003,59, 2207-2213) and these porphyrins were metallated according to the reference (A. D Adler et al, J. Inorg Nucl. Chem 1970, 32, 2443-2445). The present invention also relates to a process for the oxidation of alcohols.

### BACKGROUND OF THE INVENTION

In the prior art, supported metalloporphyrins as heterogeneous catalysts have been prepared by using different organic and inorganic solid supports such as—
1. Zeolites (P. Battioni et al, J molecular Catalysis A Chemical 109 1996,91-98)
2. Molecular sieves (Zhen Li et al, J molecular Catalysis A. Chemical 185, 2002,47-56) and
3. Polymers (M. V. Vinodu and M Padmanabhan, Proc Indian Acad Sci (Chem Sci), 1998, 5, 461-470, R A Sheldon, Metalloporphyrins in catalytic oxidations, Marcel Dekker, Inc., New York, 1994, Chapter 11; B. Meunier, Chem Rev., 1992, 92, 1411-1456, C Du, Z Li, X. Wen, J. Wu, X. Yu, M Yang and R. Xie, J. Mol. Catal A. Chem., 2004, 216, 7-12; M Moghadam, S Tangestaninejad, M. H Habibi and V Mirkhani, J. Mol. Catal A Chem 2004, 217, 9-12, S Y S. Cheng, N. Rajapakse, S. J. Rettig and B R. James, J Chem Soc. Chem Commun, 1994, 2669-2670

There is no report in the prior art literature where metalloporphyrins are encapsulated in polystyrene matrix using microencapsulation technique. Synthesis of metalloporphyrins and their use in oxidative reactions of organic substrates are of interest in porphyrin model system studies and such compounds have shown potential as industrial catalysts. Porphyrin chemistry has undergone a renaissance over the past ten years due to potential application of these compounds in the areas including drug metabolic model systems, photodynamic therapy, therapeutics for prevention of blindness, solar energy conversion, oxidative DNA cleavage, catalysis and most recently porphyrins have shown the property of binding with gold nano particles. Needless to say that it is one of the niche areas in the field of synthetic organic chemistry.

### OBJECTS OF THE INVENTION

The main object of the invention is to provide a heterogenous supported catalyst comprising of metalloporphyrin which is stable and efficient, can be easily worked up and provides better yields in application.

It is a further object of the invention to provide a process for the oxidation of alcohols using heterogenous supported metalloporphyrin catalysts and molecular oxygen oxidant in an environmentally friendly manner without loss of yield.

### SUMMARY OF THE INVENTION

Accordingly, the present invention provides a process for the preparation of polystyrene encapsulated catalyst containing metalloporphyrin of formula I: 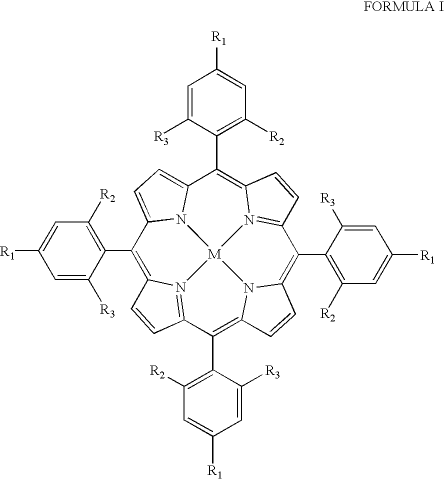
wherein R₁, R₂, and R₃ are individually selected from the group consisting of alkyl, halo, nitro and amino groups and M is a metal atom selected from the group consisting of Fe, Co, Ni, Mn, Cu, Ru, Pt and Pd, the process comprising reacting a solution of polystyrene in an organic solvent with a metalloporphyrin of formula I to obtain a dark colored solution, stirring the solution and cooling the solution, adding an alcohol drop wise to the cooled solution to obtain a thick highly viscous mass, separating the thick, highly viscous mass, and drying to obtain an encapsulated solid metalloporphyrin catalyst.

In one embodiment of the invention, the reaction of polystyrene solution with the metalloporphyrin is carried out at a maximum temperature of 60° C. and for a period ranging between 20 to 60 minutes.

In another embodiment of the invention, the dark colored solution obtained is stirred for a period in the range 30-90 minutes before cooling.

In yet another embodiment of the invention, the dark colored solution is cooled to a temperature in the range of 0 to −5° C.

In another embodiment of the invention the solvent used to prepare the polystyrene solution is selected from hydrocarbons and halogenated hydrocarbons.

In another embodiment of the invention, the organic solvent used to prepare the polystyrene solution is selected from the group consisting of chloroform dichloromethane, dichloromethane, cyclohexane and toluene.

The present invention also provides a process for aerobic oxidation of alcohols using a polystyrene encapsulated catalyst containing metalloporphyrin of formula I: 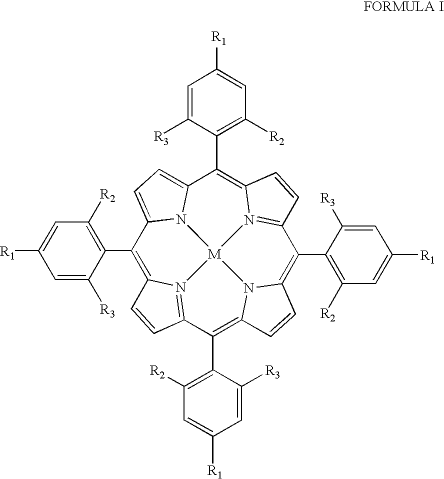
wherein R₁, R₂, and R₃ are individually selected from the group consisting of alkyl, halo, nitro and amino groups and M is a metal atom selected from the group consisting of Fe, Co, Ni, Mn, Cu, Ru, Pt and Pd, the process comprising oxidizing an alcohol over the catalyst in the presence of a sacrificial reductant and an organic solvent to obtain corresponding ketone.

In one embodiment of the invention, the oxidation is effected at a temperature in the range of at 20° C. to 70° C.

In another embodiment of the invention, the sacrificial reductant is selected from the group consisting of 2-methyl propanal, benzaldehyde, ascorbic acid and sodium borohydride.

In yet another embodiment of the invention, the organic solvent is selected from the group consisting of dichloromethane, dichloroethane, chloroform, benzene, toluene and acetonitrile.

### DETAILED DESCRIPTION OF THE INVENTION

The present invention to our knowledge is the only process for encapsulation of metalloporphyrins in polymer matrix to get solid catalysts.

The process of the invention describes preparation of immobilized metalloporphyrins, with wide applicability as versatile oxidation catalysts in the presence of molecular oxygen.

Metalloporphyrins are known to be highly efficient oxidation catalysts and immobilizing them on solid polymer support can also enhance their applicability. Such polymer supported metalloporphyrins possess several advantages over conventional homogeneous catalysts, such as, possibility of better and easy work up, recyclability and controllability of microenvironments In general case, immobilization of metal complexes on a polymer support can redound to the combination of advantages and elimination of disadvantages of homogeneous and heterogeneous catalysts.

During catalytic oxidations using metalloporphyrins, under homogeneous conditions, problems such as catalyst separation, dimerization and destruction due to self-oxidation are encountered. These disadvantages are avoided in the polystyrene supported metalloporphyrin catalyst of the invention where the microencapsulated metalloporphyrin exhibit high catalytic activity. Micro encapsulation results in immobilizing catalysts onto polymers on the basis of physical envelopment by the polymers. The catalysts are firmly anchored through the electronic interactions between the π electrons of the benzene rings of the polystyrene-based polymers and vacant orbital of catalysts.

Using micro encapsulation methodology, various substituted meso-tetra phenyl metalloporphyrins were successfully anchored on three different varieties of polystyrenes. These encapsulated catalysts were characterized by UV-Vis and diffuse reflectance FT-IR spectroscopy. These polystyrene supported metalloporphyrin catalysts thus prepared in the invention have, been demonstrated to be stable and effective in aerobic oxidation of alcohols, mimicking cytochrome P-450 dependent mono-oxygenases. The present invention provides a novel process for the preparation of the encapsulated metalloporphyrins in high yields, which overcome the disadvantages of the homogeneous metalloporphyrin catalysts. Following this viable encapsulated technique, a wide spectrum of immobilized metalloporphyrins can be synthesized.

The process of the present invention is described hereinbelow with reference to the following illustrative examples, which should not be construed to limit the scope of the present invention in any manner.

A] Examples for the Preparation of Polystyrene Supported Metalloporphyrin Catalysts

### EXAMPLE-1

Preparation of polystyrene supported [meso-tetra phenyl porphinato] cobalt (II) Polystyrene (5 g) was dissolved in 50 ml of CHCl₃ at 50° C. [Meso-tetra Phenyl porphinato] cobalt (II)(0.5 g) was added and the dark colored solution was stirred for 1 hour. Cooling of the solution to 0° C. and further addition of 50 ml of methanol (drop by drop) separates out a thick, highly viscous mass, which on drying gave a solid catalyst [meso-tetra phenyl porphinato] cobalt (II). Weight of the encapsulated catalyst: 5.42 g (98 54%). Percentage loading was determined by increase in weight and confirmed by Atomic Absorption Spectroscopy (AAS) as well as isolating unencapsulated metalloporphyrins.

### EXAMPLE-2

### Preparation of polystyrene supported chloro [meso-(2,6-dichlorophenyl) porphinato] Iron (III)

Polystyrene (5 g) was dissolved in 50 ml of CH₂Cl₂ at 40° C. Chloro [meso-(2,6-dichlorophenyl) porphinato) Iron (III) (0.5 g) was added and the dark colored solution was stirred for 1 hour. Cooling of the solution to 0° C. and further addition of 60 ml of ethanol (drop by drop) separates out a thick, highly viscous mass, which on drying gave a polystyrene supported catalyst. Weight of the encapsulated catalyst: 5.36 g (97.82%).

### EXAMPLE-3

### Preparation of polystyrene supported meso-(4-methoxyphenyl) porphinato] iron (II)

Polystyrene (5 g) was dissolved in 50 ml of CHCl₃ at 50° C. Meso-(4-methoxyphenyl) porphinato iron (II)(0.5 g) was added and the dark colored solution was stirred for 1 hour. Cooling of the solution to −5° C. and further addition of 65 ml of isopropanol (drop by drop) separates out a thick, highly viscous mass, which on drying gave polystyrene supported solid catalyst. Weight of the encapsulated catalyst; 5.46 g (99.27%).

### EXAMPLE-4

### Preparation of polystyrene supported 5,10,15,20-(4-nitro)tetra phenylporphinato nickel (II)

Polystyrene (5 g) was dissolved in 50 ml of CH₃ at 50° C. 5,10,15,20-(4-nitro)tetra phenylporphinato nickel (II)(0.5 g) was added and the dark colored solution was stirred for 1.5 hour. Cooling of the solution to 0° C. and further addition of 50 ml of methanol (drop by drop) separates out a thick, highly viscous mass, which on drying gave a solid catalyst 5,10,15,20-(4-nitro)tetra phenyl porphinato nickel (II). Weight of the encapsulated catalyst: 5.42 g (98.6%).

### EXAMPLE-5

### Preparation of polystyrene supported [meso-(2,4,6-trichlorophenyl) porphinato] iron (II)

Polystyrene (5 g) was dissolved in 50 ml of CHCl₃ at 50° C. [meso (2,4,6-trichlorophenyl) porphinato) iron (II) (0.5 g) was added and the dark colored solution was stirred for 1 hour. Cooling of the solution to 0° C. and further addition of 50 ml of ethanol (drop by drop) separates out a thick, highly viscous mass, which on drying gave a polystyrene supported catalyst. Weight of the encapsulated catalyst: 5.439 (98.7%),

### EXAMPLE-6

### Preparation of polystyrene supported [meso-(4-amino)tetra phenyl porphinato] manganese (II)

Polystyrene (5 g) was dissolved in 50 ml of CHCl₃ at 50° C. Meso-(4-amino)tetra phenyl porphinato] manganese (II) (0 5 g) was added and the dark colored solution was stirred for 1 hour. Cooling of the solution to −5° C. and further addition of 70 ml of isopropanol (drop by drop) separates out a thick, highly viscous mass, which on drying gave polystyrene supported solid catalyst. Weight of the encapsulated catalyst: 5.45 g (99.1%).

### EXAMPLE-7

### Preparation of polystyrene supported 5,10,15,20-(4-chloro)tetra phenyl porphinato cobalt (II)

Polystyrene (5 g) was dissolved in 50 ml of CH₂Cl₂ at 40° C. 5,10,15,20-(4-nitro)tetra phenyl porphinato cobalt (II)(0 5 g) was added and the dark colored solution was stirred for 1.5 hour Cooling of the solution to 0° C. and further addition of 50 ml of methanol (drop by drop) separates out a thick, highly viscous mass, which on drying gave a solid encapsulated catalyst. Weight of the encapsulated catalyst: 5.41 g (98.4%).

### EXAMPLE-8

### Preparation of polystyrene supported [meso-4-amino(2,6-dichlorophenyl) porphinato] Iron (II)

Polystyrene (5 g) was dissolved in 50 ml of CHCl₃ at 50° C. [Meso-4-amino(2,6-dichlorophenyl) porphinato] Iron (II) (0.5 g) was added and the dark colored solution was stirred for 1 hour. Cooling of the solution to 0° C. and further addition of 40 ml of ethanol (drop by drop) separates out a thick, highly viscous mass, which on drying gave a polystyrene supported catalyst. Weight of the encapsulated catalyst: 5.40 g (98.3%).

### EXAMPLE-9

### Preparation of polystyrene supported 5,10,15,20-(4-fluoro)tetra phenylporphinato copper (II)

Polystyrene (5 g) was dissolved in 50 ml of dichloroethane at 60° C. 5,10,15,20-(4-fluoro) tetra phenyl porphinato copper (II)(0 5 g) was added and the dark colored solution was stirred for 1.5 hour. Cooling of the solution to 0° C. and further addition of 45 ml of methanol (drop by drop) separates out a thick, highly viscous mass, which on drying gave a solid encapsulated catalyst. Weight of the encapsulated catalyst: 5.46 g (99.3%).

### EXAMPLE 10

### Preparation of polystyrene supported 5,10,15,20-(4-methyl)tetra phenyl porphinato manganese (II))

Polystyrene (5 g) was dissolved in 50 ml of CHCl₃ at 50° C. 5,10,15,20-(4-chloro)tetra phenyl porphinato manganese (III) (0 5 g) was added and the dark colored solution was stirred for 1 hour. Cooling of the solution to 0° C. and further addition of 60 ml of ethanol (drop by drop) separates out a thick, highly viscous mass, which on drying gave polystyrene supported catalyst. Weight of the encapsulated catalyst: 5.45 g (99.1%)

B] Examples of Oxidations of Alcohols

### EXAMPLE 11

Method for oxidation of 2-phenyl ethanol: Oxygen gas (25 ml min⁻¹) was continuously bubbled through a solution of 2-phenyl ethanol (5 mmol), polystyrene supported [meso-tetraphenyl porphinato] Cobalt (II) (corresponding to 0 056 mol % of metalloporphyrin) in 1,2-dichloroethane (20 ml) and 2-methyl propanal (15 mmol) was added to the reaction mixture at intervals of 2 hours till maximum conversion of reactant to product was achieved. The reaction mixture was vigorously stirred at 40° C. and the progress of the reaction was monitored by TLC. After stirring the reaction mixture for 5.5 hrs, it was concentrated to half. Addition of equal volume of methanol precipitated the catalyst, which was filtered and the filtrate was evaporated to dryness to give the product. The product acetophenone was purified by column chromatography and characterized by physical constants and spectroscopic data (IR, 1H-NMR and mass spectra). Yield: 0.58 g (98%),

### EXAMPLE 12

Method for oxidation of benzyl alcohol: Oxygen gas (25 ml min⁻¹) was continuously bubbled through a solution of benzyl alcohol (5 mmol), polystyrene supported [meso-(2,6-dichlorophenyl)porphinato] Iron (II) (corresponding to 0.056 mol % of metalloporphyrin) in toluene (25 ml). Ascorbic acid (15 mmol) was added to reaction mixture at intervals of 2 hours till maximum conversion of reactant to product was achieved). The reaction mixture was vigorously stirred at 65° C. and the progress of the reaction was monitored by TLC. After stirring the reaction mixture for 4 hrs, it was concentrated to half. Addition of equal volume of methanol precipitated the catalyst that was filtered and the filtrate was evaporated to dryness to give the product. The product was purified by column chromatography and characterized by physical constants and spectroscopic data (IR, 1H-NMR and mass spectra). Yield: 0.52 g (97%).

### EXAMPLE 13

Method for oxidation of menthol: Oxygen gas (25 ml min⁻¹) was continuously bubbled through a solution of menthol (5 mmol), polystyrene supported meso-(4-methoxyphenyl) porphinato] Iron (II) (corresponding to 0 056 mol % of metalloporphyrin) in chloroform (30 ml). Benzaldehyde (15 mmol) was added to the reaction mixture at intervals of 2 hours till maximum conversion of reactant to product was achieved. The reaction mixture was vigorously stirred at 50° C. and the progress of the reaction was monitored by TLC. After stirring reaction mixture for 3 hrs, it was concentrated to half. Addition of equal volume of methanol precipitated the catalyst that was filtered and filtrate was evaporated to dryness to give product. Product was purified by column chromatography and characterized by physical constants and spectroscopic data (IR, 1H-NMR and mass spectra). Yield: 0.61 g (79%),

### EXAMPLE 14

Method for oxidation of 4-nitrophenylbutanol: Oxygen gas (25 ml min⁻¹) was continuously bubbled through a solution of 4-nitrophenylbutanol (5 mmol), polystyrene supported [meso-(2,4,6-trichlorophenyl) porphinato] Iron (II) (Corresponding to 0.056 mol % of metalloporphyrin) in 1,2-dichloroethane (25 ml) and 2-methyl propanal (15 mmol) was added to the reaction mixture at intervals of 2 hours till maximum conversion of reactant to product was achieved). The reaction mixture was vigorously stirred at 70° C. and the progress of the reaction was monitored by TLC. After stirring the reaction mixture for 7 hrs, it was concentrated to half. Addition of equal volume of methanol precipitated the catalyst, which was filtered and the filtrate was evaporated to dryness to give the product. The product was purified by column chromatography and characterized by physical constants and spectroscopic data (IR, 1H-NMR and mass spectra). Yield: 0.93 g (96%),

### EXAMPLE 15

Method for oxidation of mandelic acid: Oxygen gas (25 ml min⁻¹) was continuously bubbled through a solution of mandelic acid (5 mmol), polystyrene supported (4-nitro)tetra phenylporphinato nickel (II) (corresponding to 0.056 mol % of metalloporphyrin) in toluene (30 ml). Ascorbic acid (15 mmol) was added to reaction mixture at intervals of 2.5 hours till maximum conversion of reactant to product was achieved). The reaction mixture was vigorously stirred at 65° C. and the progress of the reaction was monitored by TLC. After stirring the reaction mixture for 4 hrs, it was concentrated to half. Addition of equal volume of methanol precipitated the catalyst that was filtered and the filtrate was evaporated to dryness to give the product. The product was purified by column chromatography and characterized by physical constants and spectroscopic data (IR, 1H-NMR and mass spectra). Yield: 0.72 g (95%).

### EXAMPLE-6

Method for oxidation of 2-chlorophenyloctanol: Oxygen gas (25 ml min⁻¹) was continuously bubbled through a solution of menthol (5 mmol), polystyrene supported meso-(4-amino) tetraphenyl) porphinato]manganese (II) (corresponding to 0.056 mol % of metalloporphyrin) in chloroform (30 ml). Benzaldehyde (15 mmol) was added to the reaction mixture at intervals of 1.5 hours till maximum conversion of reactant to product was achieved. The reaction mixture was vigorously stirred at 50° C. and the progress of the reaction was monitored by TLC. After stirring the reaction mixture for 3 hrs, it was concentrated to half. Addition of equal volume of methanol precipitated the catalyst that was filtered and the filtrate was evaporated to dryness to give the product. The product was purified by column chromatography and characterized by physical constants and spectroscopic data (IR, 1H-NMR and mass spectra). Yield: 1.16 g (997%).

### EXAMPLE-7

Method for oxidation of 4-tertiary butyl cyclohexanol. Oxygen gas (25 ml min⁻¹) was continuously bubbled through a solution of 4-tertiary butyl cyclohexanol (5 mmol), polystyrene supported [meso-(2,6-dichlorophenyl) porphinato] Iron (II) (corresponding to 0.056 mol % of metalloporphyrin) in toluene (25 ml). Ascorbic acid (15 mmol) was added to reaction mixture at intervals of 2 hours till maximum conversion of reactant to product was achieved). The reaction mixture was vigorously stirred at 60° C. and the progress of the reaction was monitored by TLC. After stirring the reaction mixture for 45 hrs, it was concentrated to half. Addition of equal volume of methanol precipitated the catalyst that was filtered and the filtrate was evaporated to dryness to give the product. The product was purified by column chromatography and characterized by physical constants and spectroscopic data (IR, 1H-NMR and mass spectra). Yield: 0.74 g (96%).

### EXAMPLE-8

Method for oxidation of 4-methylcyclohexanol. Oxygen gas (25 ml min⁻¹) was continuously bubbled through a solution of 4-methylcyclohexanol (5 mmol), polystyrene supported 5,10,15,20-(4-fluoro)tetra phenylporphinato copper (II) (Corresponding to 0.056 mol % of metalloporphyrin) in chloroform (40 ml). Benzaldehyde (15 mmol) was added to the reaction mixture at intervals of 3 hours till maximum conversion of reactant to product was achieved. The reaction mixture was vigorously stirred at 60° C. and the progress of the reaction was monitored by TLC After stirring the reaction mixture for 6 hrs, it was concentrated to half. Addition of equal volume of methanol precipitated the catalyst that was filtered and the filtrate was evaporated to dryness to give the product. The product was purified by column chromatography and characterized by physical constants and spectroscopic data (IR, 1H-NMR and mass spectra). Yield: 0.54 g (96%).

### EXAMPLE-9

Method for oxidation of Benzoin, Oxygen gas (25 ml min⁻¹) was continuously bubbled through a solution of benzoin (5 mmol), polystyrene supported (4-nitro)tetra phenyl porphinato nickel (II) (corresponding to 0.056 mol % of metalloporphyrin) in ethylene dichloride (30 ml). Ascorbic acid (15 mmol) was added to reaction mixture at intervals of 2 hours till maximum conversion of reactant to product was achieved). The reaction mixture was vigorously stirred at 60° C. and the progress of the reaction was monitored by TLC. After stirring the reaction mixture for 4 hrs, it was concentrated to half. Addition of equal volume of methanol precipitated the catalyst that was filtered and the filtrate was evaporated to dryness to give the product (Benzil). The product was purified by column chromatography and characterized by physical constants and spectroscopic data (IR, 1H-NMR and mass spectra). Yield: 1.01 g (96 4%).

### EXAMPLE-10

Method for oxidation of carane diol. Oxygen gas (25 ml min⁻¹) was continuously bubbled through a solution of carane diol (5 mmol), polystyrene supported (4-nitro)tetra phenylporphinato nickel (II) (corresponding to 0.056 mol % of metalloporphyrin) in toluene (30 ml). Sodium borohydride (15 mmol) was added to reaction mixture at intervals of 2.5 hours till maximum conversion of reactant to product was achieved). The reaction mixture was vigorously stirred at 55° C. and the progress of the reaction was monitored by TLC After stirring the reaction mixture for 4 hrs, it was concentrated to half. Addition of equal volume of methanol precipitated the catalyst that was filtered and the filtrate was evaporated to dryness to give the product. The product was purified by column chromatography and characterized by physical constants and spectroscopic data (IR, 1H-NMR and mass spectra). Yield: 0.97 g (98%).

The advantages of the present invention are as follows:
(1) Owing to heterogeneous nature of supported catalyst, the destruction of catalyst due to aggegation and self oxidation is avoided resulting in to maximum catalytic effeciency.
(2) Easy work-up includes only filtration of the catalyst and removal of solvent after the reaction to get the product in high yields.
(3) These catalysts possess high turn over frequencies (TOF) without leaching in the aerobic oxidations.
(4) The catalysts are recovered quantitatively by simple filtration and reused without loss of activity,
(5) The combination of polymer supported metalloporphyrins and molecular oxygen as sole oxidant constitutes an excellent example of clean technology process for the oxidation of alcohols.

We claim:

1. A process for the preparation of polystyrene encapsulated catalyst containing metalloporphyrin of formula I: 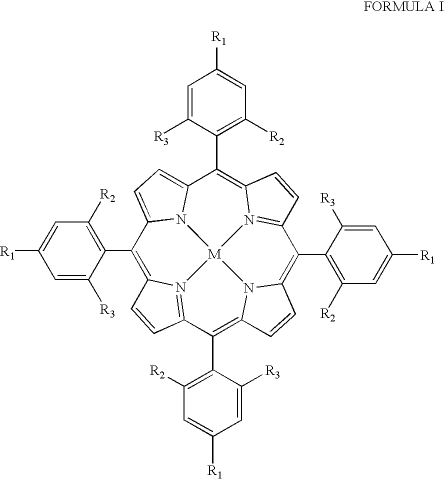
wherein R₁, R₂, and R₃ are individually selected from the group consisting of alkyl, halo, nitro and amino groups and M is a metal atom selected from the group consisting of Fe, Co, Ni, Mn, Cu, Ru, Pt and Pd, the process comprising reacting a solution of polystyrene in an organic solvent with a metalloporphyrin of formula I to obtain a dark colored solution, stirring the solution and cooling the solution, adding an alcohol drop wise to the cooled solution to obtain a thick highly viscous mass, separating the thick, highly viscous mass, and drying to obtain an encapsulated solid metalloporphyrin catalyst.

2. A process as claimed in claim 1 wherein the reaction of polystyrene solution with the metalloporphyrin is carried out at a maximum temperature of 60° C. and for a period ranging between 20 to 60 minutes.

3. A process as claimed in claim 1 wherein the dark colored solution obtained is stirred for a period in the range 30-90 minutes before cooling.

4. A process as claimed in claim 1 wherein the dark colored solution is cooled to a temperature in the range of 0 to −5° C.

5. A process as claimed in claim 1 wherein the solvent used to prepare the polystyrene solution is selected from hydrocarbons and halogenated hydrocarbons.

6. A process as claimed in claim 1 wherein the organic solvent used to prepare the polystyrene solution is selected from the group consisting of chloroform dichloromethane, dichloromethane, cyclohexane and toluene.

7. A process for aerobic oxidation of alcohols using a polystyrene encapsulated catalyst containing metalloporphyrin of formula I: 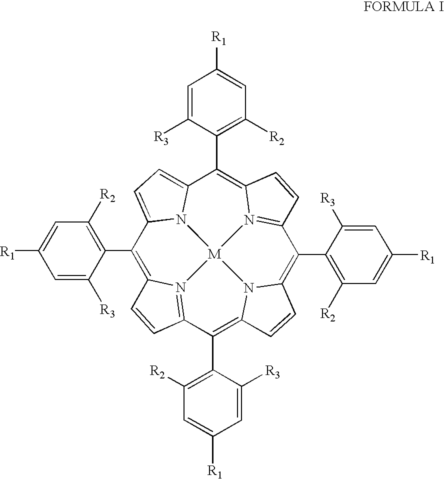
wherein R₁, R₂, and R₃ are individually selected from the group consisting of alkyl, halo, nitro and amino groups and M is a metal atom selected from the group consisting of Fe, Co, Ni, Mn, Cu, Ru, Pt and Pd, the process comprising oxidizing an alcohol over the catalyst in the presence of a sacrificial reductant and an organic solvent to obtain corresponding ketone.

8. A process as claimed in claim 7 wherein the oxidation is effected at a temperature in the range of 20° C. to 70° C.

9. A process as claimed in claim 7 wherein the sacrificial reductant is selected from the group consisting of 2-methyl propanal, benzaldehyde, ascorbic acid and sodium borohydride.

10. A process as claimed in claim 7 wherein the organic solvent is selected from the group consisting of dichloromethane, dichloroethane, chloroform, benzene, toluene and acetonitrile.

11. A process as claimed in claim 7 wherein the alcohol is selected from the group consisting of menthol, 2-phenyl ethanol and benzyl alcohol.
